(19) United States
(12) Patent Application Publication
(10) Pub. No.: US 2020/0038223 A1
(43) Pub. Date: Feb. 6, 2020

(54) CERVICAL COLLAR DEVICE

(71) Applicant: MENG-CHUN WANG, Taichung City (TW)

(72) Inventor: MENG-CHUN WANG, Taichung City (TW)

(21) Appl. No.: 16/177,908

(22) Filed: Nov. 1, 2018

(30) Foreign Application Priority Data
Aug. 6, 2018 (TW) 107210742

(51) Int. Cl. A61F 5/055

(52) U.S. Cl. CPC A61F 5/055

(57) ABSTRACT

A cervical collar device is provided, including a neck seat, including an upper neck seat portion, a lower neck seat portion and a connection portion, two opposite sides of the connection portion being respectively integrally connected to the upper neck seat portion and the lower neck seat portion, the connection portion having a seam; an adjustment device, including a lower support portion and an upper support portion which is movably connected to the lower support portion, the upper support portion being connected to the upper neck seat portion, the lower support portion being connected to the lower neck seat portion, the upper support portion having at least one first adjusting portion, the lower support portion having at least one second adjusting portion, the at least one first adjusting portion being movably positioned on the at least one second adjusting portion.

### BACKGROUND OF THE INVENTION

### Field of the Invention

The present invention relates to a cervical collar device.

### Description of the Prior Art

Generally, the injured needs to wear medical protective equipment, for example, a neck collar, a waist belt, a knee pad or an orthopedic footwear, to protect the injured body part. To meet a user's need, the neck collar usually provides a height adjusting feature, and the user can adjust the height of an upper edge of the neck collar to support a front edge of his/her chin so that the user can look forward at eyelevel.

The conventional neck collar includes a lower neck sleeve and an upper neck sleeve, and two opposite flanges of the lower neck sleeve and the upper neck sleeve overlap with each other and pivoted to each other respectively via pivoting units. The lower neck sleeve and the upper neck sleeve are designed in two-piece form, so there are many gaps between the lower neck sleeve and the upper neck sleeve, and the coverage effect is less preferable.

The present invention has arisen to mitigate and/or obviate the afore-described disadvantages.

### SUMMARY OF THE INVENTION

The major object of the present invention is to provide a cervical collar device which has a preferable coverage and can be quickly adjusted.

To achieve the above and other objects, a cervical collar device is provided, including a neck seat, including an upper neck seat portion, a lower neck seat portion and a connection portion, two opposite sides of the connection portion being respectively integrally connected to the upper neck seat portion and the lower neck seat portion, the connection portion having a seam; an adjustment device, including a lower support portion and an upper support portion which is movably connected to the lower support portion, the upper support portion being connected to the upper neck seat portion, the lower support portion being connected to the lower neck seat portion, the upper support portion having at least one first adjusting portion, the lower support portion having at least one second adjusting portion, the at least one first adjusting portion being movably positioned on the at least one second adjusting portion; wherein the upper neck seat portion, the lower neck seat portion and the connection portion are arranged on a first direction, one of the at least one first adjusting portion and the at least one second adjusting portion includes a slot which is open and extends along the first direction, the other of the at least one first adjusting portion and the at least one second adjusting portion is an arm which is disposed within the slot, and the arm abuts against at least one side wall of the slot.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of a preferred embodiment of the present invention;

FIG. 2 is a breakdown view of the preferred embodiment of the present invention;

FIG. 3 is a cross-sectional view, taken along line A-A of FIG. 1;

FIGS. 4 and 5 are partially-enlarged views showing an adjusting state of the preferred embodiment of the present invention;

FIGS. 6 and 7 are partially cross-sectional enlarged views showing an adjusting state of the preferred embodiment of the present invention; and

FIG. 8 is a stereogram of an adjustment device of the preferred embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

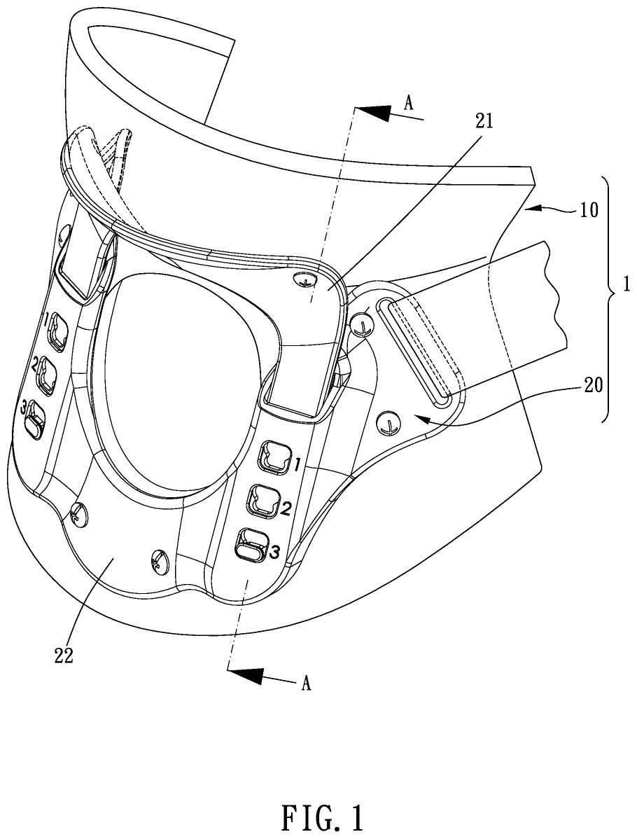

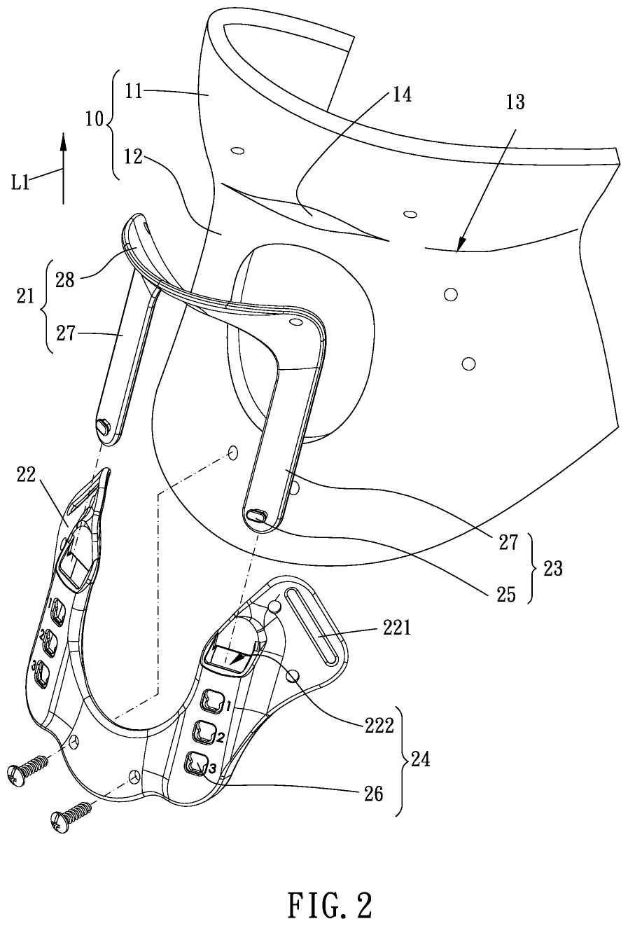

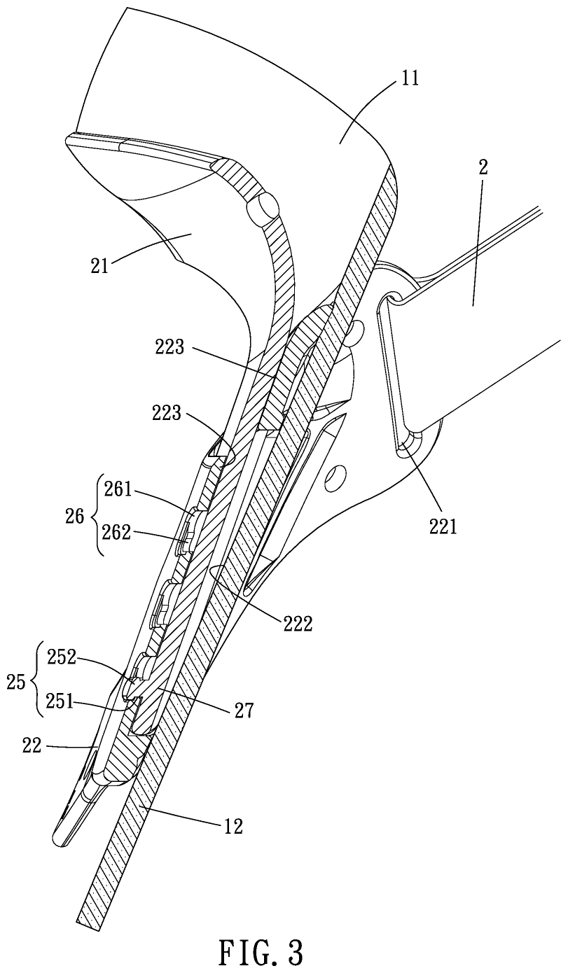

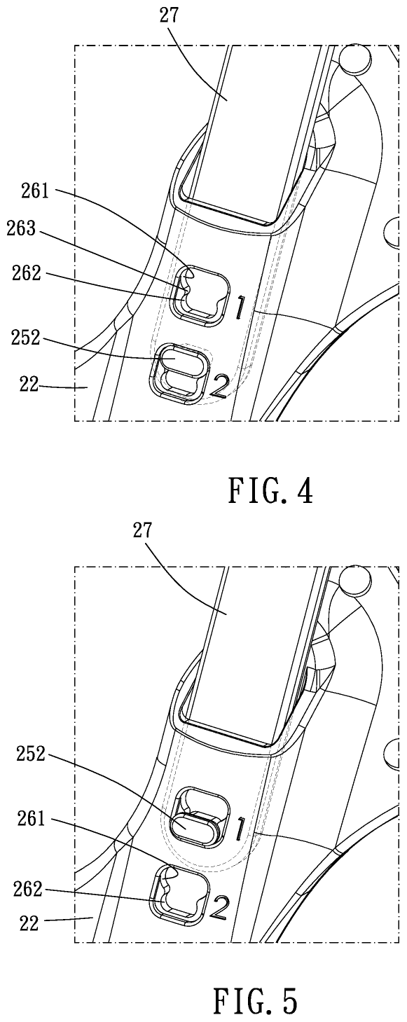

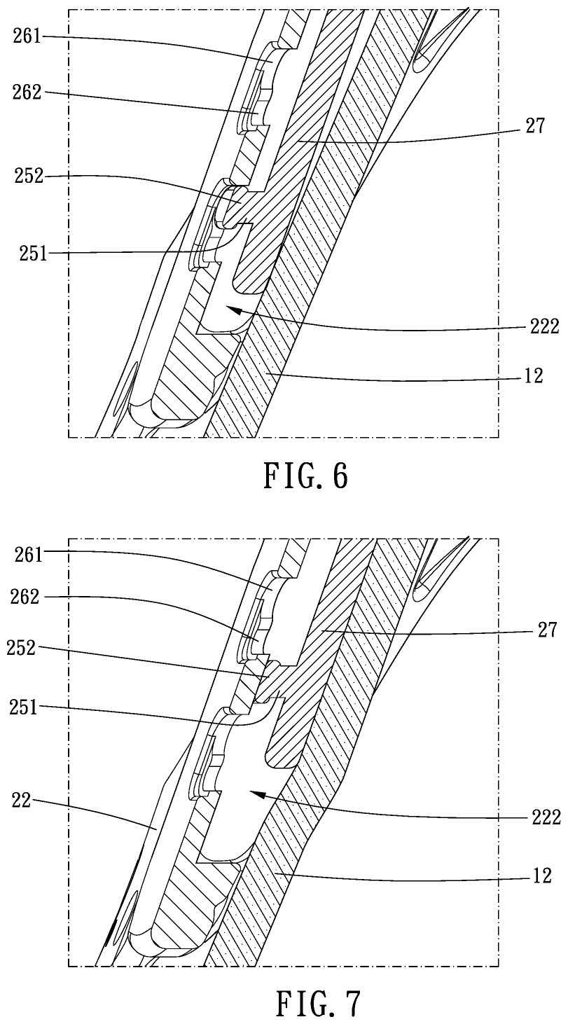

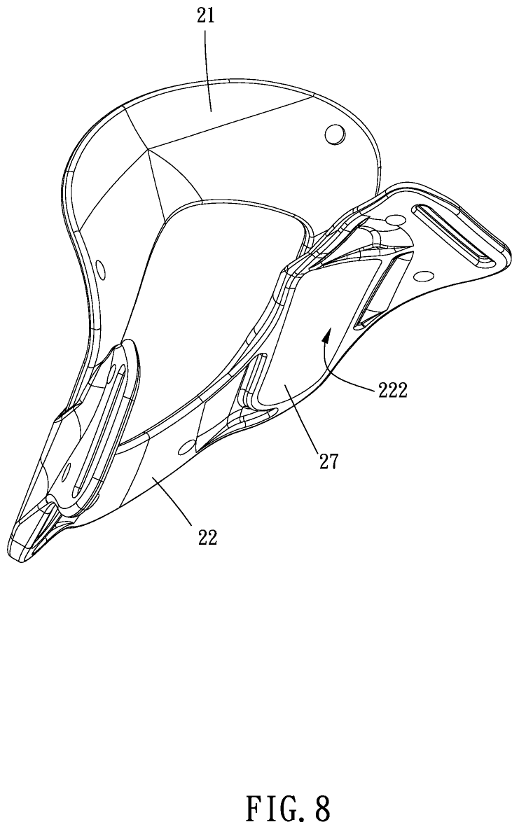

Please refer to FIGS. 1 to 8 for a preferred embodiment. A cervical collar device 1 includes a neck seat 10 and an adjustment device 20.

The neck seat 10 includes an upper neck seat portion 11, a lower neck seat portion 12 and a connection portion 13, two opposite sides of the connection portion 13 are respectively integrally connected to the upper neck seat portion 11 and the lower neck seat portion 12, and the connection portion 13 has a seam 14. The adjustment device 20 includes a lower support portion 22 and an upper support portion 21 which is movably connected to the lower support portion 22, the upper support portion 21 is connected to the upper neck seat portion 11, the lower support portion 22 is connected to the lower neck seat portion 12, the upper support portion 21 has at least one first adjusting portion 23, the lower support portion 22 has at least one second adjusting portion 24, the at least one first adjusting portion 23 is movably positioned on the at least one second adjusting portion 24, and the neck seat 10 which is integrally formed can provide better coverage and be adjustable easily. The upper neck seat portion 11, the lower neck seat portion 12 and the connection portion 13 are arranged on a first direction L1, one of the at least one first adjusting portion 23 and the at least one second adjusting portion 24 includes a slot 222 which is open and extends along the first direction L1, the other of the at least one first adjusting portion 23 and the at least one second adjusting portion 24 includes an arm 27 which is disposed within the slot 222, and the arm 27 abuts against at least one side wall 223 of the slot 222. Preferably, a number of the at least one side wall 223 is two, the two side walls 223 are disposed on two opposite sides of the slot 222, and two opposite sides of the arm 27 respectively abut against one of the two side walls 223 so that an abutting part of the arm 27 and each of the two side walls 223 forms a pivot for the arm 27 to swing relative to the slot 222.

The neck seat 10 is a seat body which is elastically deformable, for example, a sponge or a foaming sponge, which can elastically deformed and provide complete coverage and comfort.

In addition, the seam 14 is a long seam which extends along a direction lateral to the first direction so that a position of the upper neck seat portion 11 can be easily adjusted relative to the lower neck seat portion 12.

In this embodiment, each of the at least one first adjusting portion 23 is releasably buckled with one of the at least one second adjusting portion 24. In other embodiments, the at least one first adjusting portion and the at least one second adjusting portion can be connected with each other through mesh, slide fastener, stick fastener or clamp fastener. Specifically, one of the at least one first adjusting portion 23 and the at least one second adjusting portion 24 includes a plurality of engaging holes, and the other of the at least one first adjusting portion 23 and the at least one second adjusting portion 24 includes a protrusive buckle releasably engageable with one of the plurality of engaging holes. In this embodiment, the at least one second adjusting portion 24 includes the plurality of engaging holes 26, and the plurality of the engaging holes 26 are provided on one of the two side walls 223 of the slot 222.

Preferably, each of the plurality of engaging holes 26 includes a wide section 261 and a narrow section 262 which is narrower than the wide section 261, the protrusive buckle 25 includes a neck section 251 and a head section 252 which is connected to the neck section 251, and the head section 252 is detachably disposed through the wide section 261 and buckled with the narrow section 262 so as to be buckled and unbuckled quickly.

In this embodiment, a number of the at least one first adjusting portion 23 is two, each of the two first adjusting portions 23 includes the arm 27 and the protrusive buckle 263 disposed on the arm 27, the upper support portion 21 is U-shaped, and the upper support portion 21 includes the two arms 27 of the two first adjusting portions 23 and an upper prop section 28 which is connected to the two arms 27 of the two first adjusting portions 23 to provide stable support. In other embodiments, the upper support portion may also be T-shaped, and the upper support portion may have an arm and an upper prop section which is connected to the arm or have more than two arms. Each of two opposite sides of the lower support portion 22 has a buckle portion 221, two ends of a belt 2 are respectively disposed through and buckled with each of the two buckle portions 221 to cover neck, and a space which is defined by the lower support portion 22 and the belt 2 can be stably adjusted to provide preferable support. In this embodiment, the buckle portion 221 is a buckle hole; in other embodiments, the buckle portion 221 may be a Velcro, a zipper or a button. Preferably, each of two opposite sides of each of the plurality of engaging holes 26 has a protrusion 263 which extends radially, each said protrusion 263 is located between the wide section 261 and the narrow section 262, and the protrusions 263 of the two opposite sides of the plurality of engaging holes 26 abut against the neck section 251; in this embodiment, a shape of each said protrusion 263 is complementary to an outer circumference of the neck section 251 so that the protrusive buckle 25 can be stably positioned within each of the plurality of engaging holes 26.

Preferably, a width of the slot 222 expands from a bottom of the slot 222 toward an opening of the slot 222 for stable adjustment, engagement or disengagement.

Each of the at least one second adjusting portion 24 includes the slot 222, in this embodiment, each of the at least one second adjusting portion 24 and the neck seat define the slot 222 to provide a simple structure, and through pressing the neck seat 10 to make it elastically deformed, the protrusive buckle 25 can be buckled with one of the plurality of engaging holes 26. The two side walls 23 are disposed on a thickness direction of the lower support portion 22.

In actual practice, the upper neck seat portion 11 can prop a chin, the lower neck seat portion 12 props on a front chest, the upper support portion 21 can be moved relative to the lower support portion 22, and the upper neck seat portion 11 can be moved relative to the lower neck seat portion 12 via the seam 14 to adjust a height.

Given the above, the neck seat which is integrally formed can completely cover a body part which needs to be protected, and when adjusting the height, relative positions of the upper and lower neck seat portions can be quickly adjusted through the seam and the adjustment device.

While we have shown and described various embodiments in accordance with the present invention, it should be clear to those skilled in the art that further embodiments may be made without departing from the scope of the present invention.

What is claimed is:

1. A cervical collar device, including:
a neck seat, including an upper neck seat portion, a lower neck seat portion and a connection portion, two opposite sides of the connection portion being respectively integrally connected to the upper neck seat portion and the lower neck seat portion, the connection portion having a seam;
an adjustment device, including a lower support portion and an upper support portion which is movably connected to the lower support portion, the upper support portion being connected to the upper neck seat portion, the lower support portion being connected to the lower neck seat portion, the upper support portion having at least one first adjusting portion, the lower support portion having at least one second adjusting portion, the at least one first adjusting portion being movably positioned on the at least one second adjusting portion;
wherein the upper neck seat portion, the lower neck seat portion and the connection portion are arranged on a first direction, one of the at least one first adjusting portion and the at least one second adjusting portion includes a slot which is open and extends along the first direction, the other of the at least one first adjusting portion and the at least one second adjusting portion includes an arm which is disposed within the slot, and the arm abuts against at least one side wall of the slot.

2. The cervical collar device of claim 1, wherein the neck seat is a seat body which is elastically deformable.

3. The cervical collar device of claim 1, wherein the upper neck seat portion, the lower neck seat portion and the connection portion are arranged on the first direction, and the seam is a long seam which extends along a direction lateral to the first direction.

4. The cervical collar device of claim 1, wherein each of the at least one first adjusting portion is releasably buckled with one of the at least one second adjusting portion.

5. The cervical collar device of claim 1, wherein one of the at least one first adjusting portion and the at least one second adjusting portion includes a plurality of engaging holes, and the other of the at least one first adjusting portion and the at least one second adjusting portion includes a protrusive buckle releasably engageable with one of the plurality of engaging holes.

6. The cervical collar device of claim 5, wherein each of the plurality of engaging holes includes a wide section and a narrow section which is narrower than the wide section, the protrusive buckle includes a neck section and a head section which is connected to the neck section, and the head section is detachably disposed through the wide section and buckled with the narrow section.

7. The cervical collar device of claim 1, wherein a width of the slot expands from a bottom of the slot toward an opening of the slot.

8. The cervical collar device of claim 1, wherein the at least one second adjusting portion and the neck seat define the slot.

9. The cervical collar device of claim 1, wherein a number of the at least one side wall is two, the two side walls are disposed on two opposite sides of the slot, and two opposite sides of the arm respectively abut against one of the two side walls.

10. The cervical collar device of claim 6, wherein the neck seat is a seat body which is elastically deformable; the upper neck seat portion, the lower neck seat portion and the connection portion are arranged on the first direction, and the seam is a long seam which extends along a direction lateral to the first direction; each of the at least one first adjusting portion is releasably buckled with one of the at least one second adjusting portion; a width of the slot expands from a bottom of the slot toward an opening of the slot; the at least one second adjusting portion and the neck seat define the slot; a number of the at least one first adjusting portion is two, each of the two first adjusting portions includes the arm and the protrusive buckle disposed on the arm, the upper support portion is U-shaped, and the upper support portion includes the two arms of the two first adjusting portions and an upper prop section which is connected to the two arms of the two first adjusting portions; each of two opposite sides of the lower support portion has a buckle portion; a number of the at least one side wall is two, the two side walls are disposed on two opposite sides of the slot, and two opposite sides of the arm respectively abut against one of the two side walls; each of two opposite sides of each of the plurality of engaging holes has a protrusion which extends radially, each said protrusion is located between the wide section and the narrow section which is narrower than the wide section, and the protrusions of the two opposite sides of the plurality of engaging holes abut against the neck section; the plurality of engaging holes are disposed on one of the two side walls of the slot.
